(19) United States
(12) Patent Application Publication
(10) Pub. No.: US 2019/0142949 A1
(43) Pub. Date: May 16, 2019

(54) COVALENT CONJUGATES

(71) Applicant: GLAXOSMITHKLINE INTELLECTUAL PROPERTY DEVELOPMENT LIMITED, Brentford (GB)

(72) Inventors: John Alexander BROWN, Stevenage (GB); Katherine Louise Jones, Stevenage (GB); Rabinder Kumar Prinjha, Stevenage (GB); Jason Witherington, Stevenage (GB)

(21) Appl. No.: 16/244,186

(22) Filed: Jan. 10, 2019

Related U.S. Application Data
(63) Continuation of application No. 15/559,518, filed on Sep. 19, 2017.

(30) Foreign Application Priority Data
Mar. 19, 2015 (GB) 1504694.9

(51) Int. Cl. A61K 47/54; A61K 31/4709; A61K 31/4439; C07D 405/14; C07D 401/12; C07D 401/04

(52) U.S. Cl. CPC A61K 47/542; A61K 31/4709; A61K 31/4439; A61K 47/54; C07D 401/12; C07D 401/04; C07D 405/14

(57) ABSTRACT

The present invention relates to covalent conjugates of BET inhibitors and alpha amino acid esters, processes for their preparation, compositions containing them, and to their use in the treatment of various disorders in particular inflammatory and autoimmune diseases, such as rheumatoid arthritis; and cancers.

### FIELD OF THE INVENTION

The present invention relates to covalent conjugates of BET inhibitors and alpha amino acid esters, processes for their preparation, compositions containing them, and to their use in the treatment of various disorders in particular inflammatory and autoimmune diseases, such as rheumatoid arthritis; and cancers.

### BACKGROUND TO THE INVENTION

The genomes of eukaryotic organisms are highly organised within the nucleus of the cell. The long strands of duplex DNA are wrapped around an octomer of histone proteins (most usually comprising two copies of histones H2A, H2B, H3 and H4) to form a nucleosome. This basic unit is then further compressed by the aggregation and folding of nucleosomes to form a highly condensed chromatin structure. A range of different states of condensation are possible, and the tightness of this structure varies during the cell cycle, being most compact during the process of cell division. Chromatin structure plays a critical role in regulating gene transcription, which cannot occur efficiently from highly condensed chromatin. The chromatin structure is controlled by a series of post translational modifications to histone proteins, notably histones H3 and H4, and most commonly within the histone tails which extend beyond the core nucleosome structure. These modifications include acetylation, methylation, phosphorylation, ubiquitinylation, SUMOylation. These epigenetic marks are written and erased by specific enzymes, which place tags on specific residues within the histone tail, thereby forming an epigenetic code, which is then interpreted by the cell to allow regulation of gene expression.

Histone acetylation is most usually associated with the activation of gene transcription, as the modification relaxes the interaction of the DNA and the histone octomer by changing the electrostatics. In addition to this physical change, specific proteins recognise and bind to acetylated lysine residues within histones to read the epigenetic code. Bromodomains are small (˜110 amino acid) distinct domains within proteins that bind to acetylated lysine resides commonly but not exclusively in the context of histones. There is a family of around 50 proteins known to contain bromodomains, and they have a range of functions within the cell.

The BET family of bromodomain containing proteins comprises 4 proteins (BRD2, BRD3, BRD4 and BRDT) which contain tandem bromodomains capable of binding to two acetylated lysine residues in close proximity, increasing the specificity of the interaction. Numbering from the N-terminal end of each BET protein the tandem bromodomains are typically labelled Binding Domain 1 (BD1) and Binding Domain 2 (BD2) (Chung et al, J Med. Chem., 2011, 54, 3827-3838).

Inhibiting the binding of a BET protein to acetylated lysine residues has the potential to ameliorate progression of several diseases, including but not limited to, cancer (Dawson M. A. et al, Nature, 2011: 478(7370):529-33; Wyce, A. et al, Oncotarget. 2013: 4(12):2419-29), sepsis (Nicodeme E et al, Nature, 2010: 468(7327):1119-23), autoimmune and inflammatory diseases such as rheumatoid arthritis and multiple sclerosis (Mele D. A. et al, Journal of Experimental Medicine, 2013: 210(11):2181-90), heart failure (Anand P. et al, Cell, 2013: 154(3):569-82), and lung fibrosis (Tang X et al, Molecular Pharmacology, 2013: 83(1):283-293).

There exists a need for further chemical compounds that are capable of inhibiting the binding of BET proteins to acetylated lysine residues and hence have utility in the treatment of, for example, autoimmune and inflammatory diseases, and cancers. In particular, there exists a need for new approaches for generating further BET inhibitors that have improved properties over existing BET inhibitors, for example, improved potency, safety, tolerability, pharmacokinetics and/or pharmacodynamics.

### SUMMARY OF THE INVENTION

In the broadest aspect, the present invention provides a covalent conjugate of a BET inhibitor and an alpha amino acid ester, wherein the ester group of the alpha amino acid ester is hydrolysable by one or more intracellular carboxylesterases to the corresponding carboxylic acid.

The present invention utilises intracellular carboxylesterase enzymes to improve the therapeutic profile of the BET inhibitor (i.e improve potency, duration of action and/or reduce its systemic exposure). In particular, the present invention provides a new method for selectively targeting BET inhibitors to cells that express hCE-1, such as monocytes, macrophages and dendritic cells, and thus enables delivery of the BET inhibitor to those cells that are pivotal to the development and progression of numerous autoimmune and inflammatory diseases.

### BRIEF DESCRIPTION OF THE DRAWINGS

FIG. 1 shows a schematic of the hydrolysis of a covalent conjugate of the present invention.

### DETAILED DESCRIPTION OF THE INVENTION

### Definitions

As used herein, the term “bromodomain” refers to evolutionary and structurally conserved modules (approximately 110 amino acids in length) that bind acetylatedlysine residues, such as those on the N-terminal tails of histones. They are protein domains that are found as part of much larger bromodomain containing proteins (BCPs), many of which have roles in regulating gene transcription and/or chromatin remodelling. The human genome encodes for at least 57 bromodomains.

As used herein, the term “BET” refers to the bromodomain and extraterminal domain family of bromodomain containing proteins which include BRD2, BRD3, BRD4 and BRDT.

As used herein, the phrase “BET inhibitor” refers to a compound that is capable of inhibiting the binding of one or more BET family bromodomain containing proteins (e.g. BRD2, BRD3, BRD4 or BRDT) to, for example, acetylated lysine residues. Numerous BET inhibitors are disclosed in the art, such as, for example, those disclosed in WO2009/084693, WO2011/054841, WO2011/054843, WO2011/054844, WO2011/054845, WO2011/054553, WO2011/054846, WO2011/054848, WO2011/054851, WO2011/143669, WO2011/161031, WO2012/075456, WO2012/075383, WO2012/143413, WO2012/143416, WO2012/150234, WO2012/151512, WO2012/174487, WO2013/024104, WO2013/027168, WO2013/033268, WO2013/030150, WO2013/097052, WO2013/097601, WO2013/156869, WO2013/186612, WO2013/158952, WO2013/184878, WO2013/184876, WO2013/185284, WO2013/188381, WO2014/026997, WO2014/028547, WO2014/048945, WO2014/076237, WO2014/078257, WO2014/080290, WO2014/080291, WO2014/095774, WO2014/095775, WO2014/096965, WO2014/128067, WO2014/128070, WO2014/128111, WO2014/128655, WO2014/134232, WO2014/134267, US2014256706, WO2014/139324, WO2014/140076, WO2014/140077, WO2014/145051, WO2014/143768, WO2014/152029, WO2014/159837, WO2014/159392, WO2014/160873, WO2014/164596, WO2014/170350, WO2014/173241, WO2014/18929, WO2014/191894, WO2014/191896, WO2014/191906, WO2014/191911, WO2014/202578, WO2014/206345, WO2015/002754, WO2015/004533, WO2015/004534, WO2015/004075, WO2015/011084, WO2015/015318, WO2015022332, and WO2015031824, the generic and specific BET inhibitor disclosures of which are incorporated herein by reference.

As used herein, the phrase “unconjugated BET inhibitor” refers to the BET inhibitor molecule before it has been conjugated to the alpha amino acid ester either directly or indirectly through a linker molecule.

As used herein, the phrase “alpha amino acid” refers to an amino acid of general formula NH₂—CH(R)—COOH wherein R represents the side-chain of a natural alpha amino acid or an unnatural alpha amino acid.

As used herein, the phrase “natural alpha amino acid” means each form (i.e. L- and D- where possible) of the amino acids arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine and tryptophan.

As used herein, the phrase “unnatural alpha amino acid” refers to alpha amino acids of formula NH₂—CH(R)—COOH, wherein the “R” substituent is not one that exists in a natural alpha amino acid.

As used herein, the term “alkyl” refers to a saturated hydrocarbon chain, straight or branched, having the specified number of carbon atoms. For example, C₁₋₆ alkyl refers to an alkyl group having from 1 to 6 carbon atoms. Unless otherwise stated, alkyl groups are unsubstituted. The term “alkyl” includes, but is not limited to, methyl, ethyl, propyl (n-propyl and isopropyl), butyl (n-butyl, sec-butyl, isobutyl and tert-butyl), pentyl, and hexyl.

As used herein, the term “alkoxy” refers to an —O-alkyl group wherein “alkyl” is defined above.

As used herein, the term “cycloalkyl” refers to a saturated, monocyclic, hydrocarbon ring having 3 (cyclopropyl), 4 (cyclobutyl), 5 (cyclopentyl), 6 (cyclohexyl) or 7 (cycloheptyl) carbon atoms.

As used herein, the term “heterocycloalkyl” refers to a saturated or unsaturated 3 to 7 membered monocyclic ring, which must contain 1 or 2 non-carbon atoms, which are selected from nitrogen, oxygen, and sulfur. Heterocycloalkyl groups may contain one or more C(O), S(O) or SO₂ groups. However, heterocycloalkyl groups are not aromatic. Heterocycloalkyl groups containing more than one heteroatom may contain different heteroatoms. “5 or 6 membered heterocycloalkyl” refers to a saturated or unsaturated 5 or 6 membered monocyclic ring, which must contain 1 or 2 non-carbon atoms, which are selected from nitrogen, oxygen, and sulfur. Heterocycloalkyl includes, but is not limited to, pyrrolidine, piperidine, piperazine, oxetane, tetrahydrofuran, tetrahydro-2H-pyran, morpholine, morpholine-3-one, piperidin-2-one, pyrimidine-2,4(1H,3H)-dione, thiomorpholine, and thiomorpholine 1,1-dioxide.

As used herein, the term “subject” refers to an animal or human body.

As used herein, the term “treatment” refers to prophylaxis of the condition, ameliorating or stabilising the specified condition, reducing or eliminating the symptoms of the condition, slowing or eliminating the progression of the condition, and preventing or delaying reoccurrence of the condition in a previously afflicted patient or subject.

As used herein, the term “therapeutically effective amount” refers to the quantity of a covalent conjugate which will elicit the desired biological response in an animal or human body.

### STATEMENT OF THE INVENTION

In a first aspect, the present invention provides a covalent conjugate of a BET inhibitor and an alpha amino acid ester, wherein the ester group of the alpha amino acid ester is hydrolysable by one or more intracellular carboxylesterases to the corresponding carboxylic acid.

The present invention provides a general method of improving the potency or duration of action of a BET inhibitor by modification of such inhibitors through covalent conjugation with an alpha amino acid ester.

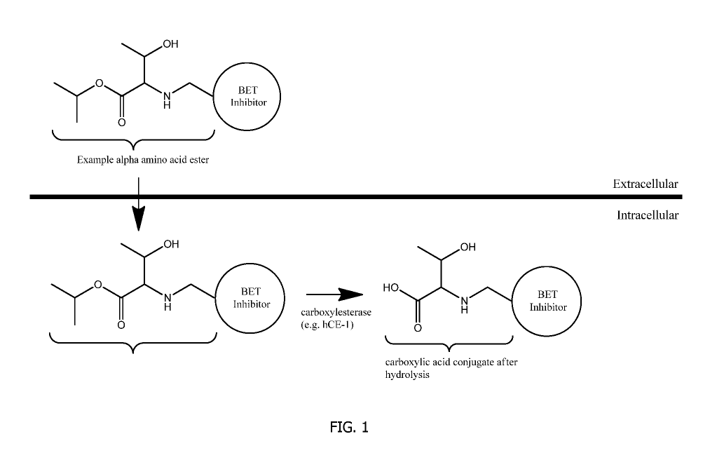

The covalent conjugates of the present invention readily penetrate through cell membranes, which is essential given that the BET family of bromodomains are intracellular proteins. Once within a cell, the alpha amino acid ester motif of the covalent conjugate is hydrolysed by a carboxylesterase enzyme to provide the corresponding carboxylic acid (carboxylic acid conjugate). The resultant carboxylic acid conjugate is charged and as a result has a reduced ability to penetrate back out of the cell. This, consequently, may lead to an increase in cellular concentration, residence time, potency or duration of action of the carboxylic acid conjugate. The schematic in FIG. 1 provides a simplistic view of the process. Even though compounds of the invention comprising an alpha amino acid ester are converted to their corresponding carboxylic acid by intracellular esterases, both the esters and their corresponding acids function as inhibitors of the BET family of bromodomain containing proteins.

The alpha amino acid ester is covalently attached to the BET inhibitor in such a way that it does not result in a significant reduction of intracellular binding activity of the BET inhibitor with its target BET protein. In general, attachment should be at a position on the molecule that is known to have little or no interaction with the target, i.e. at a position on the molecule that is not considered part of one of the binding modes that may be determined by techniques known in the art, such as X-ray crystallography. Further, the alpha amino acid ester may be attached directly to the BET inhibitor via its amino group or alpha carbon group, or may be attached through the use of a linker, such as a —(CH₂)ₙ— or —(CH₂)ₙ—O—, wherein n is 1 to 6.

In one embodiment, the present invention provides a covalent conjugate wherein the alpha amino acid ester is conjugated to the BET inhibitor such that the potency of the covalent conjugate in an in vitro binding assay is no less than 50% of the potency of the unconjugated BET inhibitor in the same assay. A suitable in vitro binding assay is the TR-FRET assay, provided herein below.

In a further embodiment, the present invention provides a covalent conjugate wherein the alpha amino acid ester is conjugated to the BET inhibitor such that the potency of the covalent conjugate in an in vitro binding assay is no less than 90% of the potency of the unconjugated BET inhibitor in the same assay. A suitable in vitro binding assay is the TR-FRET assay, provided herein below.

In a further embodiment, the present invention provides a covalent conjugate wherein the alpha amino acid ester is conjugated to the BET inhibitor such that the potency of the covalent conjugate in an in vitro binding assay is not less than the potency of the unconjugated BET inhibitor in the same assay. A suitable in vitro binding assay is the TR-FRET assay, provided herein below.

The alpha amino acid ester may be covalently attached to the BET inhibitor via the amino group of the alpha amino acid ester. Alternatively, it may be covalently attached via the alpha carbon. As stated above, a linker group may be present between the alpha amino acid ester and the BET inhibitor to facilitate the conjugation. In one embodiment, the linker is represented by the group “Q”.

In one embodiment, the alpha amino acid ester is conjugated to the BET inhibitor via the amino group of the amino acid ester and is of formula (I):

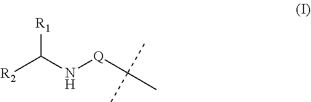

wherein Q represents —(CH₂)ₐ(O)_{b}—;
R₁ represents the side-chain of a natural or unnatural alpha amino acid and R₂ represents an ester group which is hydrolysable by one or more intracellular carboxylesterase enzymes to the corresponding carboxylic acid;
a represents 0, 1, 2 or 3; and
b represents 0 or 1, with the proviso that when b is 1, a is 2 or 3.

In a further embodiment, the alpha amino acid ester is conjugated to the BET inhibitor via the amino group of the amino acid ester and is of formula (I):

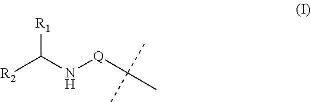

wherein Q represents —(CH₂)ₐ(O)_{b}—;
R₁ represents hydrogen, C₁₋₆alkyl, —(CH₂)_{c}cycloalkyl, —(CH₂)_{c}heterocycloalkyl, or —CR₄R₅R₆, and further wherein R₄ is hydrogen, hydroxyl, —CH₂OH, CH₂C₁₋₃alkyl, halo, —COOH, —CONH₂, 1H-imidazol-4-yl, —SH, —SeH, C₁₋₃alkyl, C₁₋₃alkoxy, phenyl, or 4-hydroxyphenyl wherein said C₁₋₃alkyl or C₁₋₃alkoxy may be optionally substituted with halo, hydroxyl, —NHC(═NH₂)NH₂, —NH₂, —COOH, —CONH₂, or —SCH₃, and R₅ and R₆ are each independently hydrogen or C₁₋₃alkyl;
R₂ represents an ester group which is hydrolysable by one or more intracellular carboxylesterase enzymes to the corresponding carboxylic acid;
a represents 0, 1, 2 or 3;
b represents 0 or 1, with the proviso that when b is 1, a is 2 or 3;
c is 0, 1 or 2.

In a further embodiment, the alpha amino acid ester is conjugated to the BET inhibitor via the alpha carbon of the amino acid ester and is of formula (II):

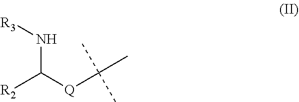

wherein Q represents —(CH₂)ₐ(O)_{b}—;
R₂ represents an ester group which is hydrolysable by one or more intracellular carboxylesterase enzymes to the corresponding carboxylic acid;
R₃ represents hydrogen, C₁₋₆alkyl or cycloalkyl;
a represents 0, 1, 2 or 3;
b represents 0 or 1, with the proviso that when b is 1, a is 2 or 3.

In a further embodiment, R₂ in the compound of formula (I) or the compound of formula (II) above represents —C(O)OCHR₇R₈ wherein R₇ is C₁₋₃alkyl or hydrogen and R₈ is C₁₋₆alkyl, cycloalkyl, heterocycloalkyl, further wherein C₁₋₆alkyl is optionally substituted with C₁₋₃alkoxy.

In a further embodiment, R₂ in the compound of formula (I) or the compound of formula (II) above represents —C(O)OR₉ wherein Re represents isopropyl, isobutyl or cyclopentyl.

In a further embodiment of the present invention, the alpha carbon of the alpha amino acid ester is in the S configuration and thus for formula (I) of formula (II) can be displayed as:

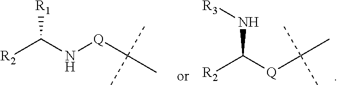

In one embodiment, the BET inhibitor when unconjugated to the alpha amino acid ester has a pIC50 of greater than 7.0 for any one of the BET proteins (BRD2, BRD3, BRD4 or BRDT) in an in vitro binding assay. An example in vitro binding assay is the TR-FRET assay, provided herein below.

There are three known intracellular human carboxylesterases (hCE-1, hCE-2 and hCE-3). Carboxyesterases hCE-2 and hCE-3 have a ubiquitous expression pattern, whereas hCE-1 is highly expressed in liver, lung and bone marrow and is, importantly, found in monocytes, macrophages and dendritic cells. In one embodiment, the covalent conjugates of the present invention may be hydrolysed by each of hCE-1, hCE-2 and hCE-3. In another embodiment, the covalent conjugates of the present invention are only hydrolysed by hCE-1 and not hCE-2 or hCE-3 and thus are selectively targeted to cells that express hCE-1, such as macrophages, monocytes and/or dendritic cells. Selective hydrolysis by hCE-1 (and thus selective targeting to cells that express hCE-1) is achieved when the nitrogen of the amino group of the alpha amino acid ester is a) not directly linked to a carbonyl group or b) not unsubstituted.

In a further embodiment, the present invention provides a covalent conjugate of a BET inhibitor and an alpha amino acid ester, wherein the alpha amino acid ester is hydrolysable by cells containing hCE-1 and not by cells that contain carboxylesterases hCE-2 and/or hCE-3, but not hCE-1.

Selectively targeting specific cell types, for example macrophages and monocytes that express hCE-1, has the potential to reduce systemic exposure of the BET inhibitor and improve safety and tolerability. Further, if retention of the BET inhibitor (in the form of the carboxylic acid conjugate) within the cell leads to improved potency or duration of action then this may enable administration of a lower dose or less frequent dosing, reducing the systemic exposure further and increasing the Therapeutic Index of the BET inhibitor.

Selection of a particular alpha amino acid ester for conjugation can also be based on its rate of hydrolysis. The alpha amino acid esters will possess different rates of hydrolysis depending on the ester group selected and, in the case of an N-linked alpha amino acid ester, the alpha carbon substituent selected. Further, the desired rate of hydrolysis will likely differ depending on the method of administration chosen for the covalent conjugate. The rate of hydrolysis of any particular alpha amino acid ester, or covalent conjugate of the present invention comprising an alpha amino acid ester, cna be determined using the “hydrolysis by hCE-1” assay outlined in the Biological Data section below. Furthermore, equivalent assays can be routinely prepared by the person skilled in the art to assess the hydrolysis of any given alpha amino acid ester, or covalent conjugate comprising such alpha amino acid ester, by a different human carboxylesterase enzyme (i.e hCE-2 or hCE-3).

The present inventors have found that for an orally administered compound, ester groups that have a slower rate of hydrolysis are desired, for example between 0.05 and 5.0, or 0.05 and 1.0, or 0.05 and 0.5, or 0.1 and 0.5, or 0.2 and 0.4 μM/min/μM (μM of covalent conjugate per minute per μM of hCE-1). As well as being present in cells of interest (e.g. monocytes, macrophages and dendritic cells depending on the target disease or condition), hCE-1 is also present in hepatocytes and therefore to ensure that a sufficient amount of the compounds makes it into circulation an ester with a slower rate of hydrolysis is desirable. In particular, the present inventors have found that covalent conjugates that possess an alpha amino acid ester that has a rate of hydrolysis of between 0.2 and 0.5 μM/min/μM have a desirable therapeutic profile that balances first pass metabolism with the enhanced properties (potency, duration of action, reduced systemic exposure, and/or increased therapeutic index) that are derived from hydrolysis of the alpha amino acid ester intracellularly.

In one embodiment of the present invention, a desirable rate of hydrolysis for an orally administered compound may be obtained if the alpha amino acid ester is of formula (I):

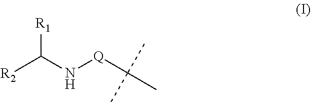

wherein R₁ represents cycloalkyl, heterocycloalkyl or —CR₄R₅R₆ wherein R₄ is hydrogen, hydroxyl, —CH₂OH, —CH₂C₁₋₃alkyl, halo, C₁₋₃alkyl, C₁₋₃alkoxy wherein said C₁₋₃alkyl or C₁₋₃alkoxy may be optionally substituted with halo or hydroxyl and R₅, and R₆ are independently hydrogen or C₁₋₃alkyl, with the proviso that at least two of R₄, R₅ and R₆ are not hydrogen; and further wherein R₂ represents —C(O)OCHR₇R₈ wherein R₇ is C₁₋₃alkyl and R₈ is C₁₋₆alkyl, cycloalkyl, heterocycloalkyl, further wherein C₁₋₆alkyl is optionally substituted with C₁₋₃alkoxy, or R₇ and R₈ together form a cycloalkyl or heterocycloalkyl group.

In a further embodiment of the present invention, a desirable rate of hydrolysis for an orally administered compound may be obtained if the alpha amino acid ester is of formula (I):

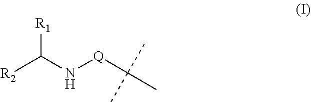

wherein R₁ represents isopropyl, sec-butyl, or —CH(CH₃)OH and R₂ represents —C(O)OR₉ wherein R₉ is isopropyl, sec-butyl, sec-pentyl, 3-pentyl, or cycloalkyl.

In a further embodiment of the present invention, a desirable rate of hydrolysis for an orally administered compound may be obtained if the alpha amino acid ester is of formula (I):

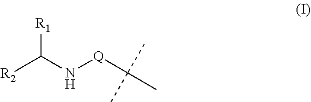

wherein R₁ represents isopropyl, sec-butyl, or —CH(CH₃)OH and R₂ represents —C(O)OR₉ wherein R₉ is isopropyl or cyclopentyl.

In another aspect of the present invention, there is provided a method for selectively targeting BET inhibitors to cells that contain hCE-1, which method comprises covalently attaching said BET inhibitor to an alpha amino acid ester that is hydrolysable by hCE-1.

In another aspect of the present invention, there is provided a method for increasing the intracellular potency of a BET inhibitor, which method comprises covalently attaching said BET inhibitor to an alpha amino acid ester that is hydrolysable by one of more carboxylesterase enzymes.

In a further aspect of the present invention, there is provided a method for reducing the systemic exposure of a BET inhibitor, which method comprises covalently attaching said BET inhibitor to an alpha amino acid ester that is hydrolysable by one or more intracellular carboxylesterase enzymes.

### Statement of Use

The covalent attachment of an alpha amino acid ester to a BET inhibitor has the potential to improve the therapeutic profile of the BET inhibitor, by reducing systemic exposure, improving potency and/or improving duration of action.

Furthermore, the selective targeting of the covalent conjugates to cells that express hCE-1, such as monocytes, macrophages and/or dendritic cells, may have therapeutic utility in the treatment of autoimmune or inflammatory diseases or conditions.

BET inhibitors may be useful in the treatment of a wide variety of acute or chronic autoimmune or inflammatory conditions such as rheumatoid arthritis, osteoarthritis, acute gout, psoriasis, systemic lupus erythematosus, pulmonary arterial hypertension (PAH), multiple sclerosis, inflammatory bowel disease (Crohn's disease and Ulcerative colitis), asthma, chronic obstructive airways disease, pneumonitis, myocarditis, pericarditis, myositis, eczema, dermatitis (including atopic dermatitis), alopecia, vitiligo, bullous skin diseases, nephritis, vasculitis, hypercholesterolemia, atherosclerosis, Alzheimer's disease, depression, Sjögren's syndrome, sialoadenitis, central retinal vein occlusion, branched retinal vein occlusion, Irvine-Gass syndrome (post cataract and post-surgical), retinitis pigmentosa, pars planitis, birdshot retinochoroidopathy, epiretinal membrane, cystic macular edema, parafoveal telengiectasis, tractional maculopathies, vitreomacular traction syndromes, retinal detachment, neuroretinitis, idiopathic macular edema, retinitis, dry eye (keratoconjunctivitis Sicca), vernal keratoconjunctivitis, atopic keratoconjunctivitis, uveitis (such as anterior uveitis, pan uveitis, posterior uveitis, uveitis-associated macular edema), scleritis, diabetic retinopathy, diabetic macula edema, age-related macular dystrophy, hepatitis, pancreatitis, primary biliary cirrhosis, sclerosing cholangitis, Addison's disease, hypophysitis, thyroiditis, type I diabetes, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's Arteritis, pyoderma gangrenosum, vasculitis with organ involvement and acute rejection of transplanted organs. The use of BET inhibitors for the treatment of rheumatoid arthritis is of particular interest.

In one embodiment, the acute or chronic autoimmune or inflammatory condition is a disorder of lipid metabolism via the regulation of APO-A1 such as hypercholesterolemia, atherosclerosis and Alzheimer's disease.

In another embodiment, the acute or chronic autoimmune or inflammatory condition is a respiratory disorder such as asthma or chronic obstructive airways disease.

In another embodiment, the acute or chronic autoimmune or inflammatory condition is a systemic inflammatory disorder such as rheumatoid arthritis, osteoarthritis, acute gout, psoriasis, systemic lupus erythematosus, multiple sclerosis or inflammatory bowel disease (Crohn's disease and ulcerative colitis).

In another embodiment, the acute or chronic autoimmune or inflammatory condition is multiple sclerosis.

In a further embodiment, the acute or chronic autoimmune or inflammatory condition is Type I diabetes.

BET inhibitors may be useful in the treatment of diseases or conditions which involve inflammatory responses to infections with bacteria, viruses, fungi, parasites or their toxins, such as sepsis, acute sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, post-surgical syndromes, sarcoidosis, Herxheimer reactions, encephalitis, myelitis, meningitis, malaria and SIRS associated with viral infections such as influenza, herpes zoster, herpes simplex and coronavirus. In one embodiment, the disease or condition which involves an inflammatory response to an infection with bacteria, a virus, fungi, a parasite or their toxins is acute sepsis.

BET inhibitors may be useful in the treatment of conditions associated with ischaemia-reperfusion injury such as myocardial infarction, cerebro-vascular ischaemia (stroke), acute coronary syndromes, renal reperfusion injury, organ transplantation, coronary artery bypass grafting, cardio-pulmonary bypass procedures, pulmonary, renal, hepatic, gastro-intestinal or peripheral limb embolism.

BET inhibitors may be useful in the treatment of fibrotic conditions such as idiopathic pulmonary fibrosis, renal fibrosis, post-operative stricture, keloid scar formation, scleroderma (including morphea), cardiac fibrosis and cystic fibrosis.

BET inhibitors may be useful in the treatment of viral infections such as herpes simplex infections and reactivations, cold sores, herpes zoster infections and reactivations, chickenpox, shingles, human papilloma virus (HPV), human immunodeficiency virus (HIV), cervical neoplasia, adenovirus infections, including acute respiratory disease, poxvirus infections such as cowpox and smallpox and African swine fever virus. In one embodiment, the viral infection is a HPV infection of skin or cervical epithelia. In another embodiment, the viral infection is a latent HIV infection.

BET inhibitors may be useful in the treatment of cancer, including hematological (such as leukaemia, lymphoma and multiple myeloma), epithelial including lung, breast and colon carcinomas, midline carcinomas, mesenchymal, hepatic, renal and neurological tumours.

BET inhibitors may be useful in the treatment of one or more cancers selected from brain cancer (gliomas), glioblastomas, Bannayan-Zonana syndrome, Cowden disease, Lhermitte-Duclos disease, breast cancer, inflammatory breast cancer, colorectal cancer, Wilm's tumor, Ewing's sarcoma, rhabdomyosarcoma, ependymoma, medulloblastoma, colon cancer, head and neck cancer, kidney cancer, lung cancer, liver cancer, melanoma, squamous cell carcinoma, ovarian cancer, pancreatic cancer, prostate cancer, sarcoma cancer, osteosarcoma, giant cell tumor of bone, thyroid cancer, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma, megakaryoblastic leukemia, acute megakaryocytic leukemia, promyelocytic leukemia, mixed lineage leukaemia, erythroleukemia, malignant lymphoma, Hodgkins lymphoma, non-Hodgkins lymphoma, lymphoblastic T-cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), NUT-midline carcinoma and testicular cancer.

In one embodiment, the cancer is a leukaemia, for example a leukaemia selected from acute monocytic leukemia, acute myelogenous leukemia, chronic myelogenous leukemia, chronic lymphocytic leukemia and mixed lineage leukaemia (MLL). In another embodiment, the cancer is NUT-midline carcinoma. In another embodiment, the cancer is multiple myeloma. In another embodiment, the cancer is a lung cancer such as small cell lung cancer (SCLC). In another embodiment, the cancer is a neuroblastoma. In another embodiment, the cancer is Burkitt's lymphoma. In another embodiment, the cancer is cervical cancer. In another embodiment, the cancer is esophageal cancer.

In another embodiment, the cancer is ovarian cancer. In another embodiment, the cancer is breast cancer. In another embodiment, the cancer is colorectal cancer.

In one embodiment, the disease or condition for which a BET inhibitor is indicated is selected from diseases associated with systemic inflammatory response syndrome, such as sepsis, burns, pancreatitis, major trauma, haemorrhage and ischaemia. In this embodiment, the BET inhibitor would be administered at the point of diagnosis to reduce the incidence of SIRS, the onset of shock, multi-organ dysfunction syndrome, which includes the onset of acute lung injury, ARDS, acute renal, hepatic, cardiac or gastro-intestinal injury and mortality. In another embodiment, the BET inhibitor would be administered prior to surgical or other procedures associated with a high risk of sepsis, haemorrhage, extensive tissue damage, SIRS or MODS (multiple organ dysfunction syndrome). In a particular embodiment, the disease or condition for which a BET inhibitor is indicated is sepsis, sepsis syndrome, septic shock and endotoxaemia. In another embodiment, the BET inhibitor is indicated for the treatment of acute or chronic pancreatitis. In another embodiment, the BET inhibitor is indicated for the treatment of burns.

In a further aspect, there is also provided a covalent conjugate of the present invention for use in therapy.

In a further aspect, there is also provided a covalent conjugate of the present invention for use in the treatment of diseases or conditions for which a bromodomain inhibitor, in particular a BET inhibitor, is indicated, including each and all of the above listed indications.

In a further aspect, there is also provided a covalent conjugate of the present invention for use in the treatment of autoimmune and inflammatory diseases, and cancer.

In a further aspect, there is also provided a covalent conjugate of the present invention for use in the treatment of rheumatoid arthritis.

In a further aspect, there is also provided a method of treatment of an autoimmune or inflammatory disease or cancer, which comprises administering to a subject in need thereof, a therapeutically effective amount of a covalent conjugate of the present invention.

In yet a further aspect, the present invention is directed to a method of treating rheumatoid arthritis, which comprises administering to a subject in need thereof, a therapeutically effective amount of a covalent conjugate of the present invention.

In a further aspect, there is provided the use of a covalent conjugate of the present invention in the manufacture of a medicament for use in the treatment of an autoimmune or inflammatory disease, or cancer.

In a further aspect, there is provided the use of a covalent conjugate of the present invention in the manufacture of a medicament for use in the treatment of rheumatoid arthritis.

### Pharmaceutical Compositions/Routes of Administration/Dosages

While it is possible that for use in therapy, the covalent conjugates of the present invention may be administered as the raw chemical, it is common to present the active ingredient as a pharmaceutical composition.

In a further aspect, there is provided a pharmaceutical composition comprising a covalent conjugate of the present invention and one or more pharmaceutically acceptable excipients.

Pharmaceutical compositions may be adapted for administration by any appropriate route, for example by the oral (including buccal or sublingual), rectal, inhaled, intranasal, topical (including buccal, sublingual or transdermal), ocular (including topical, intraocular, subconjunctival, episcleral, sub-Tenon), vaginal or parenteral (including subcutaneous, intramuscular, intravenous or intradermal) route. Such compositions may be prepared by any method known in the art of pharmacy, for example by bringing into association the active ingredient with the excipient(s).

In one aspect, the pharmaceutical composition is adapted for oral administration.

A therapeutically effective amount of a covalent conjugate of the present invention will depend upon a number of factors including, for example, the age and weight of the subject, the precise condition requiring treatment and its severity, the nature of the formulation, and the route of administration, and will ultimately be at the discretion of the attendant physician or veterinarian. In the pharmaceutical composition, each dosage unit for oral administration preferably contains from 0.01 to 1000 mg, more preferably 0.5 to 100 mg, of a covalent conjugate calculated as the free base.

### Examples

The following example covalent conjugates (with the exception of Example 10 that is an unfunctionalised BET inhibitor) have been included to illustrate, but not limit, the present invention.

| | |
|---|---|
| Example 1: (2S,3R)-isopropyl 2-(((2-(1,5- dimethyl-6-oxo-1,6-dihydropyridin-3-yl)-1- ((tetrahydro-2H-pyran-4-yl)methyl)-1H- benzo[d]imidazol-6-yl)methyl)amino)-3- hydroxybutanoate System B, 0.82 min, MH⁺ 511 | 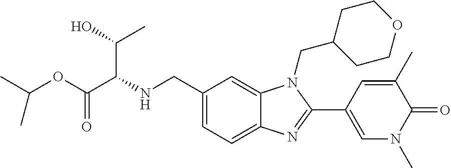 [SMILES: Cc1cc(-c2nc3ccc(CN[C@H](C(=O)OC(C)C)[C@@H](C)O)cc3n2CC2CCOCC2)cn(C)c1=O] |
| Example 2: (2S,3R)-isopropyl 2-(((2-(1,5- dimethyl-6-oxo-1,6-dihydropyridin-3-yl)-1-(((S)- tetrahydrofuran-2-yl)methyl)-1H- benzo[d]imidazol-5-yl)methyl)amino)-3- System B, 0.87 min, MH⁺ 497 | 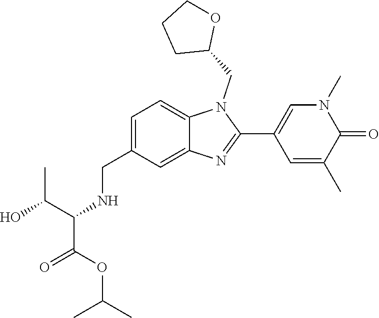 [SMILES: Cc1cc(-c2nc3cc(CN[C@H](C(=O)OC(C)C)[C@@H](C)O)ccc3n2C[C@@H]2CCCO2)cn(C)c1=O] |
| Example 3: (S)-cyclopentyl 4-methyl-2-(((2-(5- methyl-6-oxo-1,6-dihydropyridin-3-yl)-1- ((tetrahydro-2H-pyran-4-yl)methyl)-1H- benzo[d]imidazol-5-yl)methyl)amino)pentanoate LCMS (System A): t_{REF} = 0.78 min; MH⁺ 535 | 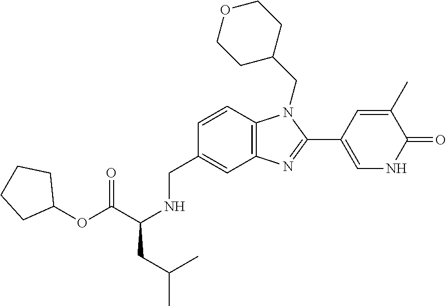 [SMILES: Cc1cc(-c2nc3cc(CN[C@@H](CC(C)C)C(=O)OC4CCCC4)ccc3n2CC2CCOCC2)c[nH]c1=O] |
| Example 4: (2S,3R)-cyclobutyl 2-(((2-(1,5- dimethyl-6-oxo-1,6-dihydropyridin-3-yl)-1-((S)-1- methoxpropan-2-yl)-1H-benzo[d]imidazol-6- yl)methyl)amino)-3-hydroxybutanoate System B, 0.93 min, MH⁺ 497 | 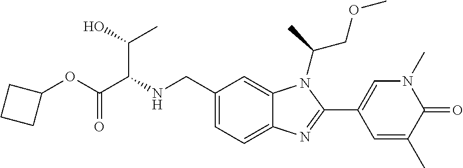 [SMILES: COC[C@H](C)n1c(-c2cc(C)c(=O)n(C)c2)nc2ccc(CN[C@H](C(=O)OC3CCC3)[C@@H](C)O)cc21] |
| Example 5: (2S,3R)-isobutyl 2-(((2-(1,5- dimethyl-6-oxo-1,6-dihydropyridin-3-yl)-1- methyl-1H-benzo[d]imidazol-5-yl)methyl)amino)- 3-hydroxybutanoate System B, 0.89 min, MH⁺ 441 | 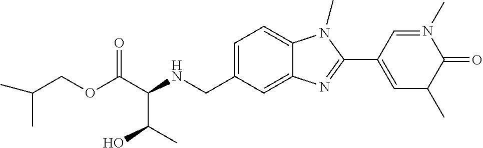 |
| Example 6: (2S,3R)-isopropyl 2-(((1- (cyclopropylmethyl)-2-(1,5-dimethyl-6-oxo-1,6- dihydropyridin-3-yl)-1H-benzo[d]imidazol-6- yl)methyl)amino)-3-hydroxybutanoate System B, 0.94 min, MH⁺ 467 | 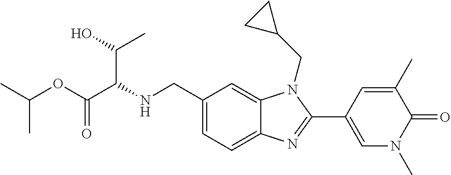 |
| Example 7: (2S,3R)-cyclobutyl 2-(((2-(1,5- dimethyl-6-oxo-1,6-dihydropyridin-3-yl)-1- ((tetrahydro-2H-pyran-4-yl)methyl)-1H- benzo[d]imidazol-5-yl)methyl)amino)-3- hydroxybutanoate System B, 0.90 min, MH⁺ 523 | 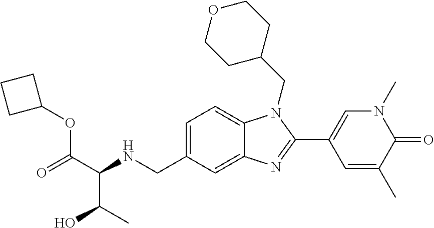 [SMILES: Cc1cc(-c2nc3cc(CN[C@H](C(=O)OC4CCC4)[C@@H](C)O)ccc3n2CC2CCOCC2)cn(C)c1=O] |
| Example 8: (2S,3R)-cyclobutyl 2-(((1-(1,3- dimethoxypropan-2-yl)-2-(1,5-dimethyl-6-oxo- 1,6-dihydropyridin-3-yl)-1H-benzo[d]imidazol-5- yl)methyl)amino)-3-hydroxybutanoate System B, 0.95 min, MH⁺ 529 | 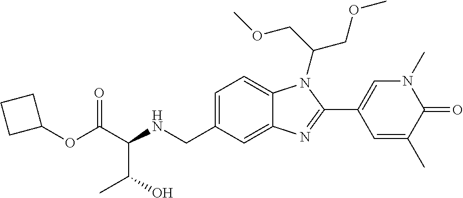 [SMILES: COCC(COC)n1c(-c2cc(C)c(=O)n(C)c2)nc2cc(CN[C@H](C(=O)OC3CCC3)[C@@H](C)O)ccc21] |
| Example 9: (S)-isopropyl 2-(((2-(1,5-dimethyl- 6-oxo-1,6-dihydropyridin-3-yl)-1-((S)-1- hydroxypropan-2-yl)-1H-benzo[d]imidazol-5- yl)methyl)amino)-3-methylbutanoate System A, 0.54 min, MH⁺ 469 | 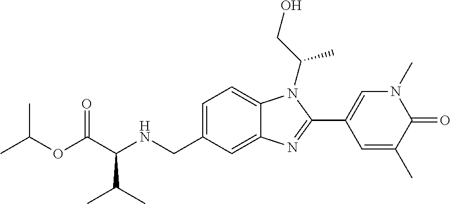 [SMILES: Cc1cc(-c2nc3cc(CN[C@H](C(=O)OC(C)C)C(C)C)ccc3n2[C@@H](C)CO)cn(C)c1=O] |
| Example 10: 3-methyl-5-(1-((tetrahydro-2H- pyran-4-yl)methyl)-1H-benzo[d]imidazol-2- yl)pyridin-2(1H)-one System A, 0.54 min, MH⁺ 324 | 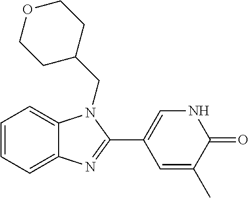 [SMILES: Cc1cc(-c2nc3ccccc3n2CC2CCOCC2)c[nH]c1=O] |

Examples 1 to 10 above may be prepared according to the following general reaction schemes.

There is provided a process for the preparation of Examples 1 to 10, which process comprises cyclisation of a compound of formula (III):

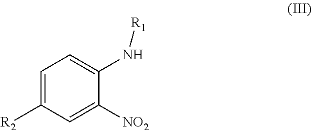

wherein R₁ and R₂ are as they appear above in any of Examples 1 to 10 in the table above. For example, a compound of formula (III) could be dissolved in a solvent mixture such as ethanol/water, then treated with an aldehyde of formula (VI), wherein Rₐ is hydrogen or methyl, in the presence of sodium dithionite and heated at a suitable temperature for an appropriate time to give, after purification, Examples 1 to 10.

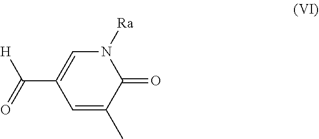

There is provided a process for the preparation of a compound of formula (III), which process comprises the nucleophilic functionalisation of a compound of formula (V):

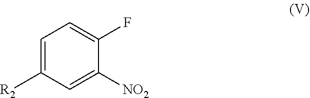

wherein R₂ is as shown in any of Examples 1 to 8 in the table above. For example, a compound of formula (V) could be dissolved in a solvent such as tetrahydrofuran then treated with a suitable amine containing R₁ as shown in any of Examples 1 to 10 in the table above in the presence of a suitable base such as triethylamine. The mixture would then be heated at a suitable temperature for an appropriate time to give, after purification, compounds of the formula (III).

There is provided a process for the preparation of a compound of formula (V), which process comprises the reductive amination of the compound of formula (VI):

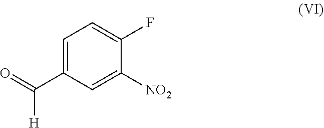 [SMILES: O=Cc1ccc(F)c([N+](=O)[O-])c1]

Wherein the compound of formula (VI) is dissolved in a suitable solvent such as dicloromethane to which is added an appropriately functionalised amine and an additive such as acetic acid. The mixture would be stirred at an appropriate temperature for a specific time prior to the addition of a reducing agent such as sodium triacetoxyborohydride. The mixture would be stirred for an appropriate time to give, after purification, compounds of formula (V) wherein R₂ is as shown in any of Examples 1 to 10 in the table above.

The following Example (Example 11) details the preparation of an additional covalent conjugate between an alpha amino acid ester and a BET inhibitor, wherein the BET inhibitor is a different chemotype to those of Examples 1 to 10.

### Example 11: (S)-cyclopentyl 2-((4-((2S,4R)-1-acetyl-4-((5-cyanopyridin-2-yl)amino)-2-methyl-1,2,3,4-tetrahydroquinolin-6-yl)benzyl)amino)-4-methylpentanoate

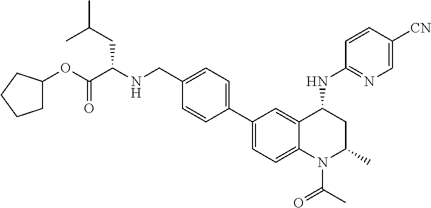 [SMILES: CC(=O)N1c2ccc(-c3ccc(CN[C@@H](CC(C)C)C(=O)OC4CCCC4)cc3)cc2[C@H](Nc2ccc(C#N)cn2)C[C@@H]1C]

A round bottom flask was charged with 6-(((2S,4R)-1-acetyl-6-bromo-2-methyl-1,2,3,4-tetrahydroquinolin-4-yl)amino)nicotinonitrile (For a preparation see intermediate 3, 220 mg, 0.571 mmol), (S)-(4-(((1-(cyclopentyloxy)-4-methyl-1-oxopentan-2-yl)amino)methyl)phenyl)boronic acid, 4-methylbenzenesulphonic acid salt, (For a preparation see intermediate 1318 mg, 0.629 mmol), potassium carbonate (395 mg, 2.86 mmol), Toluene (5 mL) and Ethanol (5.00 mL). To the stirred mixture was added palladium tetrakis (33.0 mg, 0.029 mmol) and the system degassed with nitrogen. The vessel was heated to reflux for 3 hours under a blanket of nitrogen. The mixture was cooled to room temperature and allowed to stand overnight. The volatiles were removed in vacuo to give an orange solid. The solid was dissolved in a 1:1 EtOAc/water mixture. The layers were mixed and separated before the organics were washed with brine, passed through a hydrophobic frit and concentrated in vacuo to give an orange oil. The sample was loaded in dichloromethane and purified by Biotage SP4 SNAP 25 g silica (Si) using a gradient of 0-60% ethyl acetate-cyclohexane over 20 CV. The appropriate fractions were combined and evaporated in vacuo before being dried under a stream of nitrogen to give the required product (S)-cyclopentyl 2-((4-((2S,4R)-1-acetyl-4-((5-cyanopyridin-2-yl)amino)-2-methyl-1,2,3,4-tetrahydroquinolin-6-yl)benzyl)amino)-4-methylpentanoate (266 mg, 0.448 mmol, 78% yield), as an off-white solid. System A, MH+=594 at 0.94 min.

### Intermediate 1: (S)-(4-(((1-(cyclopentyloxy)-4-methyl-1-oxopentan-2-yl)amino)methyl)phenyl)boronic Acid, 4-methylbenzenesulphonic Acid Salt

A round bottom flask was charged with (4-formylphenyl)boronic acid (Aldrich, 300 mg, 2.001 mmol), dichloromethane (DCM) (10 mL) and (S)-cyclopentyl 2-amino-4-methylpentanoate 4-methylbenzenesulfonate (For a preparation see intermediate 2, 751 mg, 2.021 mmol). To the stirred mixture under a blanket of nitrogen was added sodium triacetoxyborohydride (1.27 g, 5.99 mmol) portionwise over a period of 20 minutes. The mixture was stirred at room temperature for 2 hours. The reaction mixture was diluted with DCM and poured into 1M HCl. The layers were mixed and separated before the aqueous was carefully neutralised (pH7) through addition of solid NaHCO₃. The aqueous was extracted (×2) with DCM, the organics combined, passed through a hydrophobic frit and concentrated in vacuo to give the crude title compound as a white foam. Used at this purity in subsequent reactions. System A, MH+=334 at 0.74 min.

### Intermediate 2: (S)-cyclopentyl 2-amino-4-methylpentanoate 4-methylbenzenesulfonate

A round bottom flask was charged with (S)-2-amino-4-methylpentanoic acid (5 g, 38.1 mmol), cyclohexane (100 mL), tosic acid monohydrate (9.43 g, 49.6 mmol) and cyclopentanol (35 mL, 386 mmol). A Dean-Stark condensor was fitted and the mixture warmed to 130° C. to effect complete dissolution. The mixture was stirred at this temperature over the weekend before being allowed to stand at room temperature for 7 days. The precipitated solid was isolated by filtration and washed sequentially with cyclohexane and ethyl acetate. The solid was dried in vacuo to give the title compound as a white solid. ¹H NMR (400 MHz, METHANOL-d₄) δ 7.62-7.79 (m, 2H), 7.25 (d, J=7.83 Hz, 2H), 5.15-5.42 (m, 1H), 3.97 (t, J=6.97 Hz, 1H), 2.39 (s, 3H), 1.42-2.10 (m, 11H), 1.02 (d, J=7.09 Hz, 6H).

### Intermediate 3: 6-(((2S,4R)-1-acetyl-6-bromo-2-methyl-1,2,3,4-tetrahydroquinolin-4-yl)amino)nicotinonitrile

1-((2S,4R)-4-amino-6-bromo-2-methyl-3,4-dihydroquinolin-1(2H)-yl)ethanone (For a preparation see intermediate 4, 5.74 g, 20.27 mmol) was divided into 3 portions and each was dissolved in 10 ml of NMP. To each solution was added 1.86 g of 6-chloronicotinonitrile and 3.5 ml of DIPEA before the reaction mixtures were each heated at 200° C. for 2 hrs in a 20 ml microwave vial.

The reaction mixtures were cooled to r.t. and combined before being diluted with ethyl acetate (200 ml) and water (100 ml). The organic layer was extracted and aqueous further extracted with further portions of ethyl acetate (3×50 ml). The combined organic layers were dried (MgSO₄) and concentrated to give 20.27 g crude brown oil (containing NMP). This was purified by chromatography on SiO₂ (RediSep 3300 g cartridge, eluting with 10-100% ethyl acetate/cyclohexane over 10 CVs) to give 6-(((2S,4R)-1-acetyl-6-bromo-2-methyl-1,2,3,4-tetrahydroquinolin-4-yl)amino)nicotinonitrile (7.47 g, 16.48 mmol, 81% yield) as a yellow foamy solid. System A, MH+=385/387 at RT=0.98 min.

### Intermediate 4: 1-((2S,4R)-4-amino-6-bromo-2-methyl-3,4-dihydroquinolin-1(2H)-yl)ethanone

Isopropyl ((2S,4R)-1-acetyl-6-bromo-2-methyl-1,2,3,4-tetrahydroquinolin-4-yl)carbamate (for a preparation see WO02012/143415A1, 25.0 g, 67.7 mmol) was added to a cold (ice/water bath) suspension of aluminium chloride (34.3 g) in DCM (450 ml). The suspension, then solution was stirred at ˜0° C. for 30 min, before addition of triethylamine (113 ml) in methanol (60 ml). The mixture was diluted with DCM, saturated aqueous sodium hydrogen carbonate (˜500 ml) added and the mixture treated with a solution of Rochelle's salt (113 g) in water (˜21). The biphasic suspension was manually stirred at intervals over ˜30 min—majority of solid had dissolved. The phases were seperated, the aqueous extracted with DCM (×3) and the combined organic phases washed with water and then brine. The solution was dried with magnesium sulphate, filtered and reduced to dryness in vacuo to give a beige gum (˜20 g). The gum was triturated with diethyl ether, the solid isolated by filtration, washed with ether and dried in vacuo to give a white solid (6.11 g). The combined filtrate and washings were reduced to dryness under vacuum and then further dried in vacuo. The residual gum was retriturated with diethyl ether to give a white solid. The solid was isolated by filtration and washed with diethyl ether to give a white solid (1.95 g). The combined filtrate and washings were reduced to dryness in vacuo and the gummy residue dissolved in hot cyclohexane. the solution was allowed to cool to ambient temperature and left at this temperature over ˜2 h. The solid which formed was isolated by filtration, washed with cyclohexane and dried in vacuo to give white solid N18680-88-4 (5.89 g). The batches were combined to give the title compound as a white solid (13.95 g, 72.8% yield). System A, (MH⁺−NH₃)=267 at RT=0.49 min.

### Instrument Details

### NMR

¹H NMR spectra were recorded in either CDCl₃, DMSO-d₆ or MeOD-d₄ on either a Bruker DPX 400 or Bruker Avance DRX, Varian Unity 400 spectrometer or JEOL Delta all working at 400 MHz. The internal standard used was either tetramethylsilane or the residual protonated solvent at 7.25 ppm for CDCl₃ or 2.50 ppm for DMSO-d₆ or 3.31 for MeOD-d₄.

### LCMS

### System A

Column: 50 mm×2.1 mm ID, 1.7 m Acquity UPLC BEH C18
Flow Rate: 1 mL/min.

### Temp: 40° C.

UV detection range: 210 to 350 nm
Mass spectrum: Recorded on a mass spectrometer using alternative-scan positive and negative mode electrospray ionisation
Solvents: A: 0.1% v/v formic acid in water
B: 0.1% v/v formic acid acetonitrile

| | Time (min.) | A % | B % |
|---|---|---|---|
| Gradient: | 0 | 97 | 3 |
| | 1.5 | 0 | 100 |
| | 1.9 | 0 | 100 |
| | 2.0 | 97 | 3 |

### System B

Column: 50 mm×2.1 mm ID, 1.7 m Acquity UPLC BEH C₁₈
Flow Rate: 1 mL/min.

### Temp: 40° C.

UV detection range: 210 to 350 nm
Mass spectrum: Recorded on a mass spectrometer using alternative-scan positive and negative mode electrospray ionisation
Solvents: A: 10 mM ammonium bicarbonate in water adjusted to pH10 with ammonia solution
B: acetonitrile

| | Time (min.) | A % | B % |
|---|---|---|---|
| Gradient: | 0 | 99 | 1 |
| | 1.5 | 3 | 97 |
| | 1.9 | 3 | 97 |
| | 2.0 | 0 | 100 |

### System C

Column: 50 mm×2.1 mm ID, 1.7 m Acquity UPLC CSH C₁₈
Flow Rate: 1 mL/min.

### Temp: 40° C.

UV detection range: 210 to 350 nm
Mass spectrum: Recorded on a mass spectrometer using alternative-scan positive and negative mode electrospray ionisation
The solvents employed were:
A=0.1% v/v solution of Trifluoroacetic Acid in Water.
B=0.1% v/v solution of Trifluoroacetic Acid in Acetonitrile.

| | Time (min.) | A % | B % |
|---|---|---|---|
| Gradient: | 0 | 95 | 5 |
| | 1.5 | 5 | 95 |
| | 1.9 | 5 | 95 |
| | 2.0 | 95 | 5 |

### Biological Data

### Time Resolved Fluorescence Resonance Energy Transfer (TR-FRET) Assay

Binding was assessed using a time resolved fluorescent resonance energy transfer binding assay. This utilises a 6 His purification tag at the N-terminal of the proteins as an epitope for an anti-6 His antibody labeled with Europium chelate (PerkinElmer AD0111) allowing binding of the Europium to the proteins which acts as the donor fluorophore. A small molecule, high affinity binder of the bromodomain BRD4 has been labeled with Alexa Fluor647 (Reference Compound X) and this acts as the acceptor in the FRET pair.

### Reference Compound X: 4-((Z)-3-(6-((5-(2-((4S)-6-(4-chlorophenyl)-8-methoxy-1-methyl-4H-benzo[f][1,2,4]triazolo[4,3-a][1,4]diazepin-4-yl)acetamido)pentyl)amino)-6-oxohexyl)-2-((2E,4E)-5-(3,3-dimethyl-5-sulfo-1-(4-sulfobutyl)-3H-indol-1-ium-2-yl)penta-2,4-dien-1-ylidene)-3-methyl-5-sulfoindolin-1-yl)butane-1-sulphonate)

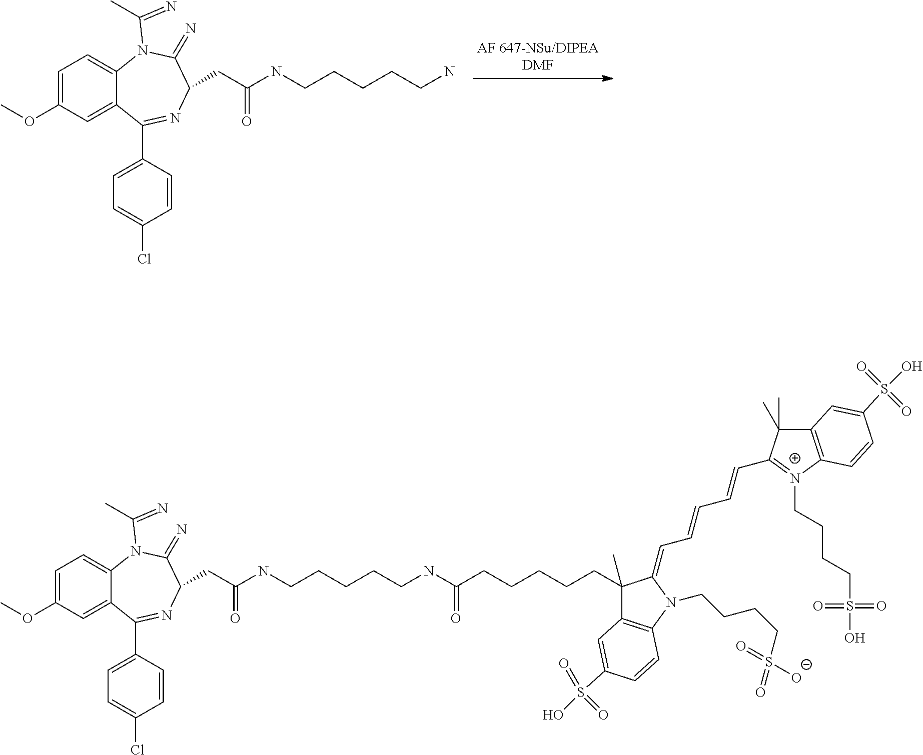 [SMILES: COc1ccc2c(c1)C(c1ccc(Cl)cc1)=N[C@@H](CC(=O)NCCCCCN)C(=N)N2C(C)=N ; COc1ccc2c(c1)C(c1ccc(Cl)cc1)=N[C@@H](CC(=O)NCCCCCNC(=O)CCCCCC1(C)/C(=C/C=C/C=C/C3=[N+](CCCCS(=O)(=O)O)c4ccc(S(=O)(=O)O)cc4C3(C)C)N(CCCCS(=O)(=O)[O-])c3ccc(S(=O)(=O)O)cc31)C(=N)N2C(C)=N]

To a solution of N-(5-aminopentyl)-2-((4S)-6-(4-chlorophenyl)-8-methoxy-1-methyl-4H-benzo[f][1,2,4]triazolo[4,3-a][1,4]diazepin-4-yl)acetamide (for a preparation see Reference Compound J, WO2011/054848A1, 1.7 mg, 3.53 μmol) in DMF (40 μl) was added a solution of AlexaFluor647-ONSu (2.16 mg, 1.966 μmol) also in DMF (100 μl). The mixture was basified with DIPEA (1 μl, 5.73 μmol) and agitated overnight on a vortex mixer. The reaction mixture was evaporated to dryness. The solid was dissolved in acetonitrile/water/acetic acid (5/4/1, <1 ml) filtered and was applied to a Phenomenex Jupiter C18 preparative column and eluted with the following gradient (A=0.1% trifluoroacetic acid in water, B=0.1% TFA/90% acetonitrile/10% water): Flow rate=10 ml/min., AU=20/10 (214 nm): 5-35%, t=0 min: B=5%; t=10 min: B=5%; t=100 min: B=35%; t=115 min: B=100% (Sep. grad: 0.33%/min)

The major component was eluted over the range 26-28% B but appeared to be composed of two peaks. The middle fraction (F1.26) which should contain “both” components was analysed by analytical HPLC (Spherisorb ODS2, 1 to 35% over 60 min): single component eluting at 28% B.

Fractions F1.25/26&27 were combined and evaporated to dryness. Transferred with DMF, evaporated to dryness, triturated with dry ether and the blue solid dried overnight at <0.2 mbar: 1.54 mg.

Analytical HPLC (Sphersisorb ODS2, 1 to 35% B over 60 min): MSM10520-1: [M+H]⁺ (obs): 661.8/—corresponding with M−29. This equates to [(M+2H)/2]⁺ for a calculated mass of 1320.984 which is M−29. This is a standard occurrence with the Alexa Fluor 647 dye and represents a theoretical loss of two methylene groups under the conditions of the mass spectrometer.

### Assay Principle:

In the absence of a competing compound, excitation of the Europium causes the donor to emit at λ618 nm which excites the Alexa labelled bromodomain binding compound leading to an increased energy transfer that is measurable at λ647 nM. In the presence of a sufficient concentration of a compound that can bind these proteins, the interaction is disrupted leading to a quantifiable drop in fluorescent resonance energy transfer.

The binding of Examples 1 to 11 to Bromodomain BRD4 was assessed using mutated proteins to detect differential binding to Binding Domain 1 (BD1) on the bromodomain. These single residue mutations in the acetyl lysine binding pocket greatly lower the affinity of the fluoroligand (Reference Compound X) for the mutated domain (>1000 fold selective for the non-mutated domain). Therefore in the final assay conditions, binding of the fluoroligand to the mutated domain cannot be detected and subsequently the assay is suitable to determine the binding of compounds to the single non-mutated bromodomain.

### Protein Production:

Recombinant Human Bromodomain [BRD4 (Y390A)] was expressed in E colicells (pET15b vector) with a 6-His tag at the N-terminal. The His-tagged Bromodomain pellet was resuspended in 50 mM HEPES (pH7.5), 300 mM NaCl, 10 mM imidazole & 1 μl/ml protease inhibitor cocktail and extracted from the E. coli cells using sonication and purified using a nickel sepharose high performance column, the proteins were washed and then eluted with a linear gradient of 0-500 mM imidazole with buffer 50 mM HEPES (pH7.5), 150 mM NaCl, 500 mM imidazole, over 20 column volumes. Final purification was completed by Superdex 200 prep grade size exclusion column. Purified protein was stored at −80° C. in 20 mM HEPES pH 7.5 and 100 mM NaCl. Protein identity was confirmed by peptide mass fingerprinting and predicted molecular weight confirmed by mass spectrometry.

### Protocol for Bromodomain BRD4, BD1 Mutant Assay:

All assay components were dissolved in buffer composition of 50 mM HEPES pH7.4, 50 mM NaCl, 5% Glycerol, 1 mM DTT and 1 mM CHAPS. The final concentration of bromodomain proteins were 10 nM and the Alexa Fluor647 ligand was at Kd. These components were premixed and 5 μl of this reaction mixture was added to all wells containing 50 nl of various concentrations of test compound or DMSO vehicle (0.5% DMSO final) in Greiner 384 well black low volume microtitre plates and incubated in dark for 30 minutes at rt. 5 μl of detection mixture containing 1.5 nM final concentration anti-6His Europium chelate was added to all wells and a further dark incubation of at least 30 minutes was performed. Plates were then read on the Envision platereader, (λex=317 nm, donor λem=615 nm; acceptor λem=665 nm; Dichroic LANCE dual). Time resolved fluorescent intensity measurements were made at both emission wavelengths and the ratio of acceptor/donor was calculated and used for data analysis. All data was normalized to the mean of 16 high (inhibitor control—Example 11 of WO 2011/054846A1) and 16 low (DMSO) control wells on each plate. A four parameter curve fit of the following form was then applied:

y=a+((b−a)/(1+(10̂x/10̂c)̂d)

Where ‘a’ is the minimum, ‘b’ is the Hill slope, ‘c’ is the pIC₅₀ and ‘d’ is the maximum.

### Results:

All of Examples 1 to 11 were tested in the above BRD4 assay and were found to have a pICso in the range of 5.8 to 7.3 in the BRD4 BD1 assay. Example 3 and Example 10 had pIC50 s of 6.1 and 6.4 respectively.

Measurement of LPS Induced MCP-1 Production from Human Whole Blood

Activation of monocytic cells by agonists of toll-like receptors such as bacterial lipopolysaccharide (LPS) results in production of key inflammatory mediators including MCP-1. Such pathways are widely considered to be central to the pathophysiology of a range of auto-immune and inflammatory disorders. Blood is collected in a tube containing Sodium heparin (Leo Pharmaceuticals) (10 units of heparin/mL of blood). 96-well compound plates containing 1 μL test sample in 100% DMSO were prepared (two replicates on account of donor variability). 130 μL of whole blood was dispensed into each well of the 96-well compound plates and incubated for 30 min at 37° C., 5% CO₂. 10 μL of lipopolysaccharide (from Salmonella typhosa; L6386) made up in PBS (200 ng/mL final assay concentration) was added to each well of the compound plates. The plates were then placed in the humidified primary cell incubator for 18-24 hours at 37° C., 5% CO₂. 140 μL of PBS was added to all wells of the compound plates containing blood. The plates were then sealed and centrifuged for 10 mins at 2500 rpm. 25 μL of cell supernatant was placed in a 96-well MSD plate pre-coated with human MCP-1 capture antibody. The plates were sealed and placed on a shaker at 600 rpm for 1 hour (r.t). 25 μL of Anti-human MCP-1 antibody labelled with MSD SULFO-TAG™ reagent is added to each well of the MSD plate (stock 50× was diluted 1:50 with Diluent 100, final assay concentration is 1 μg/mL). The plates were then re-sealed and shaken for another hour before washing with PBS. 150 μL of 2×MSD Read Buffer T (stock 4×MSD Read Buffer T was diluted 50:50 with de-ionised water) was then added to each well and the plates read on the MSD Sector Imager 6000. Concentration response curves for each compound were generated from the data and an IC₅₀ value was calculated.

### Results:

All of Examples 1 to 11, except Example 5, were tested in the above assay and were found to have a pIC₅₀ in the range of 5.6 to 8.2. Example 3 and Example 10 had pIC50 s of 7.1 and 5.6 respectively.

Hydrolysis by hCES-1

Hydrolysis of ESM-containing BET inhibitors by carboxylesterase 1 (CES1) is one aspect of delivering a targeted molecule. Rates of hydrolysis of Examples 1 to 9 and 11 by recombinant human CES1 were determined using an HPLC assay. Recombinant human CES1 (Gly18-Glu563, bearing a polyhistidine tag at the C-terminus) expressed in human cells and purified to homogeneity was obtained from Novoprotein, Summit, N.J., USA (catalogue number C450). Reactions were run in 384 well plates at 20° C. in a buffer of 50 mM sodium phosphate pH 7.5/100 mM NaCl. Assays used a fixed concentration of test compound (50 μM) and CES1 (50 nM) and a time course of the reaction was obtained by stopping samples at increasing times by addition of formic acid to lower the pH. Stopped samples were subsequently analysed by HPLC to resolve product acid from unhydrolysed ester, using a 50×2 mm C18 5 μM reversed-phase column (Phenomenex Gemini) at a flow rate of 1 ml/min using a gradient of acetonitrile in water, containing 0.1% formic acid. Chromatography was monitored using absorbance at 300 nm wavelength. The % of product formed was determined using integrated peak areas and used to determine the initial rate of the reaction. The specific activity of the CES1 against each test compound under these conditions (in units of μM/min/μM) was obtained by dividing the initial rate of the reaction by the CES1 concentration.

### Results:

All of Examples 1 to 11, except Example 10 that does not possess an alpha amino acid ester, had rates of hydrolysis of between 0.1 and 5.0 (μM of test compound hydrolysed per minute per μM of CES1) in the above assay.

1. A covalent conjugate of a BET inhibitor and an alpha amino acid ester, wherein the ester group of the alpha amino acid ester is hydrolysable by one or more intracellular carboxylesterases to the corresponding carboxylic acid, and wherein the alpha amino acid ester possesses a rate of hydrolysis of between 0.2 to 0.5 μM/min/μM.

2. The covalent conjugate according to claim 1, wherein the alpha amino acid ester is conjugated to the BET inhibitor such that the potency of the covalent conjugate in an in vitro binding assay is no less than 50% of the potency of the unconjugated BET inhibitor in the same assay.

3. The covalent conjugate according to claim 2, wherein the alpha amino acid ester is conjugated to the BET inhibitor such that the potency of the covalent conjugate in an in vitro binding assay is at least as high as that of the unconjugated BET inhibitor in the same assay.

4. The covalent conjugate according to claim 1, wherein the alpha amino acid ester is conjugated to the BET inhibitor via the amino group of the amino acid ester and is of formula (I): 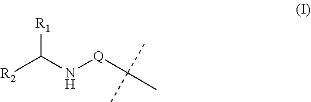
wherein Q represents —(CH₂)ₐ(O)_{b}—;
R₁ represents the side-chain of a natural or unnatural alpha amino acid;
R₂ represents an ester group which is hydrolysable by one or more intracellular carboxylesterase enzymes to the corresponding carboxylic acid;
a represents 0, 1, 2 or 3;
b represents 0 or 1, with the proviso that when b is 1, a is 2 or 3.

5. The covalent conjugate according to claim 4, wherein R₁ represents hydrogen, C₁₋₆alkyl, —(CH₂)_{c}cycloalkyl, —(CH₂)_{c}heterocycloalkyl, or —CR₄R₅R₆, and further wherein R₄ is hydrogen, hydroxyl, —CH₂OH, —CH₂C₁₋₃alkyl, halo, —COOH, —CONH₂, 1H-imidazol-4-yl, —SH, —SeH, C₁₋₃alkyl, C₁₋₃alkoxy, phenyl, or 4-hydroxyphenyl wherein said C₁₋₃alkyl or C₁₋₃alkoxy may be optionally substituted with halo, hydroxyl, —NHC(═NH₂)NH₂, —NH₂, —COOH, —CONH₂, or —SCH₃, and R₅ and R₆ are each independently hydrogen or C₁₋₃alkyl, and c is 0, 1 or 2.

6. The covalent conjugate according to claim 1, wherein the alpha amino acid ester is conjugated to the BET inhibitor via the alpha carbon of the amino acid ester and is of formula (II): 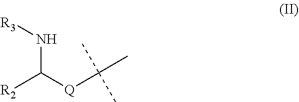
wherein Q represents —(CH₂)ₐ(O)_{b}—;
R₂ represents an ester group which is hydrolysable by one or more intracellular carboxylesterase enzymes to the corresponding carboxylic acid;
R₃ represents hydrogen, C₁₋₆alkyl or cycloalkyl;
a represents 0, 1, 2 or 3;
b represents 0 or 1, with the proviso that when b is 1, a is 2 or 3.

7. The covalent conjugate according to claim 4, wherein R₂ represents —C(O)OCHR₇R₈ wherein R₇ is C₁₋₃alkyl or hydrogen and R₈ is C₁₋₆alkyl, cycloalkyl, heterocycloalkyl, wherein said C₁₋₆alkyl is optionally substituted with C₁₋₃alkoxy, or R₇ and R₈ together form cycloalkyl or heterocycloalkyl.

8. The covalent conjugate according to claim 4, wherein R₂ represents —C(O)OR₉ wherein R₉ represents isopropyl, isobutyl or cyclopentyl.

9. The covalent conjugate according to claim 4, wherein a is 1 and b is 0.

10. The covalent conjugate according to claim 1, wherein the alpha amino acid ester is hydrolysable by cells containing hCE-1 and not by cells that contain carboxylesterases hCE-2 and/or hCE-3, but not hCE-1.

11. The covalent conjugate according to claim 10, wherein the alpha amino acid ester is of formula (I): 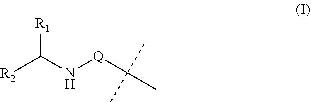
wherein R₁ represents cycloalkyl, heterocycloalkyl or —CR₄R₅R₆ wherein R₄ is hydrogen, hydroxyl, —CH₂OH, —CH₂C₁₋₃alkyl, halo, C₁₋₃alkyl, C₁₋₃alkoxy wherein said C₁₋₃alkyl or C₁₋₃alkoxy may be optionally substituted with halo or hydroxyl and R₅ and R₆ are independently hydrogen or C₁₋₃alkyl, with the proviso that at least two of R₄, R₅ and R₆ are not hydrogen, and R₂ represents —C(O)OR₉ wherein R₉ is isopropyl, sec-butyl, sec-pentyl, 3-pentyl, or cycloalkyl.

12. The covalent conjugate according to claim 1, wherein the alpha carbon of the alpha amino acid ester is in the S-configuration.

13. The covalent conjugate according to claim 1, wherein the BET inhibitor prior to conjugation to the alpha amino acid ester has a pIC50 greater than 7.0 for any one of the BET proteins in an in vitro binding assay.

14. The covalent conjugate according to claim 13, wherein the BET protein is BRD4.
